(19) United States
(12) Patent Application Publication
(10) Pub. No.: US 2025/0350819 A1
(43) Pub. Date: Nov. 13, 2025

(54) IMAGING ASSEMBLY WITH TILTABLE HOUSING

(71) Applicant: Med Smart Hub Ltd., Caesarea Industrial Park (IL)

(72) Inventors: Kfir Solomon, Zoran (IL); Gil Bachar, Tel Aviv (IL)

(73) Assignee: Med Smart Hub Ltd., Caesarea Industrial Park (IL)

(21) Appl. No.: 19/274,841

(22) Filed: Jul. 21, 2025

Related U.S. Application Data
(63) Continuation of application No. 18/064,268, filed on Dec. 11, 2022.

(51) Int. Cl. H04N 23/51; A61B 1/00; A61B 1/313; A61B 34/30

(52) U.S. Cl. CPC H04N 23/51; A61B 1/0008; A61B 1/3132; A61B 2034/302

(57) ABSTRACT

An imaging assembly includes a shaft which extends from a housing, the shaft including a distal tip, an imaging device located in the shaft or in the housing, a controller for operating the imaging device, a skin adhering fastening member coupled to the housing, and an actuator coupled to the housing and operative to tilt the housing about a joint about one or more rotation axes.

### FIELD OF THE INVENTION

The present invention relates generally to minimally invasive surgery, and particularly to an imaging assembly, which is capable of being tilted to provide improved imaging.

### BACKGROUND OF THE INVENTION

In minimally invasive surgery, there are often several small incisions (such as a primary port and ancillary ports) made into the body to insert surgical tools, insufflation devices, endoscopes, or other viewing devices. There are many advantages in reducing the number of incision points to as few as possible, such as reducing trauma to the patient, reducing the incidence of infection, improving recovery time, and decreasing cosmetic damage. The incisions may be made with a trocar, which is a guide with a sharp tip.

One of the first steps during a laparoscopic surgical procedure involves insufflation of the abdomen with nitrogen or carbon dioxide gas. The resulting expansion of the abdomen reduces the risk of injury to the contents of the abdomen during subsequent insertion of the ports and also allows the surgeons more freedom and space to manipulate instruments and perform the surgery.

Laparoscopic surgery is generally performed with only one source of visualization, namely, the camera at the tip of the laparoscope. However, in order to minimize risk of injury to the patient, it is preferable to observe the exit ports of all cannulas every time an instrument is inserted or withdrawn. Such observation currently requires that the camera on the tip of the laparoscope be directed toward a particular port. This would then result in the loss of visualization of the surgical field, which interrupts the surgical procedure and interrupts the use of the surgical instruments until the surgical field can again be visualized with the laparoscope.

Thus an improved imaging assembly is clearly needed.

### SUMMARY OF THE INVENTION

The present invention seeks to provide an improved imaging assembly with tilting capability, as is described more in detail hereinbelow.

There is thus provided in accordance with a non-limiting embodiment of the present invention an imaging assembly including a shaft which extends from a housing, the shaft including a distal portion, an imaging device located in the shaft or in the housing, a controller for operating the imaging device, a skin adhering fastening member coupled to the housing, and an actuator coupled to the housing and operative to tilt the housing about a joint about one or more rotation axes.

In accordance with a non-limiting embodiment of the present invention the actuator is operative to move the housing linearly along one or more translation axes.

In accordance with a non-limiting embodiment of the present invention the distal portion of the shaft is formed with one or more gas-inlet apertures, such that gas can flow through the gas-inlet apertures and flow past electronic components in the shaft. The housing may be coupled by a conduit to a vacuum source.

In accordance with a non-limiting embodiment of the present invention the skin adhering fastening includes a vacuum pad coupled to a vacuum source.

In accordance with a non-limiting embodiment of the present invention a position sensor and/or inclination sensor may be coupled to the housing.

### BRIEF DESCRIPTION OF THE DRAWINGS

The present invention will be understood and appreciated more fully from the following detailed description, taken in conjunction with the drawing in which:

FIG. 1 is a simplified illustration of an imaging assembly, constructed and operative in accordance with a non-limiting embodiment of the present invention.

### DETAILED DESCRIPTION

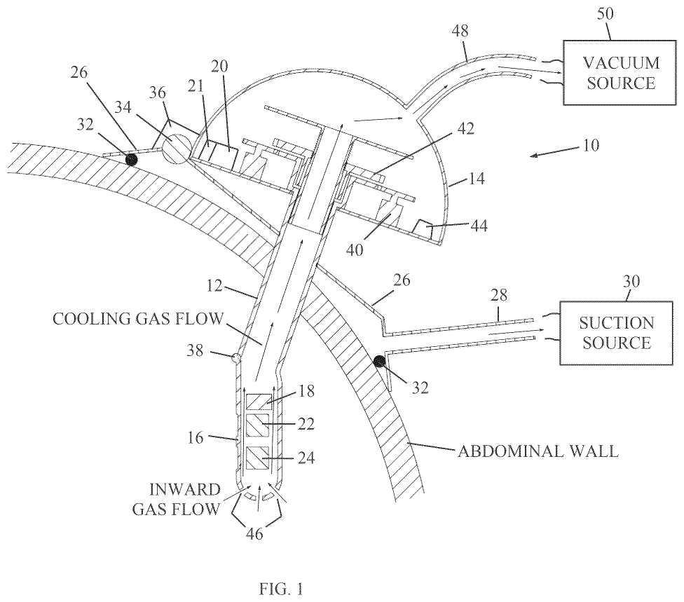

Reference is now made to FIG. 1, which illustrates an imaging assembly 10, constructed and operative in accordance with a non-limiting embodiment of the present invention.

The imaging assembly 10 includes a shaft 12, which extends from a housing 14. Shaft 12 may extend perpendicularly from housing 14, or at other non-perpendicular angles. Shaft 12 may include a distal portion 16, which may be a pointed cutting blade, in which case shaft 12 serves as a trocar which may be used to puncture skin. Alternatively, distal portion 16 may be blunt, in which case shaft 12 may enter through a separately made incision.

The imaging assembly 10 may include an imaging device 18, such as but not limited to, a camera, ultrasound sensor or other suitable imaging modality sensor. The imaging device 18 may be located at or near the distal portion 16 of shaft 12 and may view the internal portion of the patient by means of one or more optical elements. For example, optical elements may include an illumination source 22 (located in shaft 12, but could alternatively be located in housing 14) that can generate light to illuminate the area to be imaged, and light modification elements 24, such as one or more lenses or filters. In such a case, shaft 12 may be made from an optically transparent material, such as but not limited to, polymethyl methacrylate (PMMA). The distal portion 16 of shaft 12 may be beveled to disperse the lighting at any desired angle.

Alternatively, imaging device 18 may be located in housing 14 and may view the internal portion of the patient by means of light guides, mirrors, etc. A controller 20 for operating imaging device 18 may be located in housing 14, shaft 12 or externally in an external control unit. The controller 20 may transmit and receive information via a communication unit 21.

The imaging assembly 10 may include a skin adhering fastening member 26, such as but not limited to, a vacuum pad (or cup or other similar element, the terms being used interchangeably), adhesive pad or other attachment means.

In the non-limiting illustrated embodiment, skin adhering fastening member 26 is a vacuum pad with a vacuum inlet 28, and the vacuum may be supplied by a suction source 30 (e.g., vacuum pump) coupled to vacuum inlet 28. The suction source 30 may continuously control the vacuum supplied to adhere the imaging assembly 10 to the skin, and can lower or shut off the vacuum to enable quick removal of the assembly and quick repositioning to another place with renewed application of vacuum. Skin adhering fastening member 26 may include a seal 32 to seal the vacuum pad to the skin and prevent loss of suction.

The skin adhering fastening member 26 may be coupled to housing 14 by means of a joint 34. Joint 34 may be a gimbaled or multiple-degree-of-freedom joint so that housing 14 can pivot with respect to skin adhering fastening member 26 in more than one rotation angle, such as a rotation angle in the plane of the drawing and another rotation angle away from the plane of the drawing. An actuator 36, such as but not limited to, a servomotor, may be coupled to housing 14, which can tilt the housing 14 about joint 34. Additionally, the actuator 36 can move housing 14 linearly along one or more axes. Thus, actuator 36, in one embodiment, can move housing 14 in at least 4 degrees of freedom: two degrees of freedom in translation (such as along X-Y axes or other axes) and two degrees of freedom in rotation (such as about X-Y axes or other axes). Actuator 36 may be located at or near joint 34 or may alternatively be located in housing 14 or skin adhering fastening member 26.

In the illustrated embodiment, shaft 12 includes a swivel joint 38 so that the distal portion 16 of shaft 12 may be rotated about swivel joint 38 to a desired orientation. The proximal portion of shaft 12, which is located in housing 14, may be coupled to a shaft actuator 40, such as but not limited to, a motor coupled to shaft 12 through a gear train 42. Shaft actuator 40 can tilt shaft 12 about swivel joint 38 and can rotate shaft 12 about the longitudinal axis of shaft 12.

Accordingly, the housing 14 can move in multiple degrees of freedom, including movement in rotation and/or translation, and the distal portion 16 of shaft 12 may be rotated about swivel joint 38 to a desired rotational orientation. This provides the surgeon with significantly enhanced capability of capturing images at multiple orientations, and can maximize the quality of the images captured by the imaging device and reduce or eliminate the need for cropping the images. Position and/or inclination sensors 44 may be provided which send feedback to the controller 20 or to a display and provide position and orientation information to the surgeon.

In accordance with a non-limiting embodiment of the present invention, the distal portion 16 of shaft 12 may be formed with one or more gas-inlet apertures 46 (of any size and shape). Housing 14 may be coupled by a conduit 48 to a dedicated vacuum source 50 (or alternatively to the suction source 30). In this manner, gas, such as carbon dioxide. which has been introduced into the viewed area (such as the abdominal area) in a surgical procedure, can be flow through gas-inlet apertures 46 and flow past the electronic components in shaft 12 (such as illumination source 22, imaging device 18, and others) and cool them by convection. The convection may be natural convection as the gas rises through shaft 12 and flows into housing 14, preferably exiting through conduit 48. Gas can rise due to, but not limited to, pressure differences between CO2 in the internal abdominal region and outside environment. If vacuum is applied by vacuum source 50, then the convection cooling is enhanced by becoming forced convection. Accordingly, the invention provides a way of exploiting available gas to cool the electronic components of the device.

What is claimed is:

1. A method for cooling an imaging assembly comprising:
providing a shaft which extends from a housing, said shaft being formed with a lumen that extends from a distal portion of said shaft towards a proximal portion of said shaft, wherein said shaft is formed with gas-inlet apertures that are in fluid communication with said lumen;
providing an imaging device located in said lumen in said shaft; and
causing said shaft to be introduced into a body cavity so that carbon dioxide, which has been introduced into said body cavity before said shaft has been introduced in said body cavity, is free to flow through said gas-inlet apertures into said lumen and flow past said imaging device, thereby cooling said imaging device, wherein one of said gas-inlet apertures is located at a distal tip of said distal portion of said shaft along a central longitudinal axis of said distal portion, and others of said gas-inlet apertures are located traverse to the central longitudinal axis of said distal portion.

2. The method according to claim 1, further comprising tilting said housing about a joint about one or more rotation axes.

3. The method according to claim 1, wherein said distal portion of said shaft is rotatable about a swivel joint, and further comprising tilting or rotating said distal portion of said shaft about said swivel joint.

4. The method according to claim 1, further comprising using a skin adhering fastening member coupled to said housing to adhere said housing to a portion of a patient.

5. The method according to claim 1, wherein said imaging device comprises an illumination source and further comprising using said imaging device to illuminate a portion of said body cavity.

6. The method according to claim 1, further comprising using a position sensor coupled to said housing to sense a position of the housing.

7. The method according to claim 1, further comprising using an inclination sensor coupled to said housing to sense an inclination of the housing.

8. The method according to claim 1, further comprising using a vacuum to increase convection cooling of the carbon dioxide by becoming forced convection.
